(19) United States
(12) Patent Application Publication
(10) Pub. No.: US 2008/0160087 A1
(43) Pub. Date: Jul. 3, 2008

(54) Gel preparation for oral administration

(76) Inventors: Masaki Ishibashi, Higashikagawa-shi (JP); Mitsuru Endo, Higashikagawa-shi (JP); Yasushi Miwa, Higashikagawa-shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/010,741

(22) Filed: Jan. 29, 2008

(30) Foreign Application Priority Data
Jul. 13, 2005 (JP) 204552/2005

(51) Int. Cl. A61K 9/14; A61K 31/401; A61K 31/366; A61K 31/22

(52) U.S. Cl. 424/488; 514/548; 514/423; 514/460

(57) ABSTRACT

Provided for use in medical care settings is a gel preparation for oral administration to patients with hyperlipemia or hypercholesterolemia, wherein the ingestibility of an HMG-CoA reductase inhibitor, also known as a statin-series compound, has been improved in dosage forms of tablets, granules and liquids. A pharmaceutical composition in a gel form, comprising an HMG-CoA reductase inhibitor or a pharmaceutically acceptable salt thereof, a gelling agent, a polymer compound, a buffering agent, a preservative, a sweetening agent, a base and water, wherein the bad tastes of the statin-series compound are disguised, syneresis during storage is less likely, form retention is good, removal from containers is easy, and the ingestibility has been improved.

### TECHNICAL FIELD

The present invention relates to a gel preparation comprising an HMG-CoA reductase inhibitor, intended for administration to patients with hyperlipemia or hypercholesterolemia in need for oral administration of an HMG-CoA reductase inhibitor.

### BACKGROUND ART

Gel preparations for oral administration are suitable for patients having difficulty in swallowing compared with conventional solid preparations such as tablets and capsules. Additionally, these gel preparations are advantageous over liquid preparations in that the risk for aspiration is minimal, and that the bad tastes, such as bitter tastes and harsh tastes, of pharmacologically active ingredients thereof are mitigated.

Currently commercially available dosage forms for drugs belonging to the HMG-CoA reductase inhibitor family include, for example, tablets, capsules, fine granules, oral liquids and the like. These drugs are frequently administered to elderly, bedridden or tube feeding patients. The patients suffer age-related impairments in their ability to swallow, which tend to be accompanied by symptoms such as dry mouth. Generally, solid preparations are relatively difficult to swallow, whereas liquid preparations pose problems associated with aspiration and choking during swallowing. Because the recipient of an HMG-CoA reductase inhibitor used to treat hyperlipemia, a lifestyle-related disease, over several months to several years, or even longer times is often a bedridden patient, a patient with dysphagia, or an tube feeding patient, there is a demand for a gel preparation having physical properties such as viscoelasticity and liquid thickness in a dosage form that does not clog the throat, does not induce reflex vomiting, is easily swallowable, and permits painless ingestion by the patient. However, no research has been conducted into a gel preparation for oral administration comprising an HMG-CoA reductase inhibitor.

### DISCLOSURE OF THE INVENTION

Compounds belonging to the HMG-CoA reductase inhibitor family are generically called statin-series compounds, most of which are unstable in acidic aqueous solutions and suspensions, and hence must be prepared at pH levels of 7 or higher. On the other hand, because alkaline preparations at pH levels of 11 or higher are likely to cause adverse reactions such as stomatitis in taking, a gel preparation of an HMG-CoA reductase inhibitor for oral administration must be prepared at pH levels between 7 and 10. Hence, a gel preparation comprising an HMG-CoA reductase inhibitor, and adjusted to a pH levels between 7 and 10, was investigated. When agar or κ carrageenan, both commonly used pharmaceutical additives, was used as the gelling agent, the gel preparation obtained exhibited unstable physical properties such as considerable syneresis. On the other hand, if xanthan gum or ι carrageenan is added for suppressing syneresis with reducing the content ratio of agar or κ carrageenan, the gel preparation obtained lacks gel strength and exhibits poor form retention; the preparation collapses in the oral cavity at the time of removal from its containers and during ingestion, the bad tastes, such as bitter tastes and harsh tastes, of the HMG-CoA reductase inhibitor are intensely perceived, these preparations are not suitable for taking orally.

Amid this situation, the present inventors diligently investigated to obtain a gel preparation for oral administration comprising an HMG-CoA reductase inhibitor as the primary ingredient, wherein gel formation during manufacturing is not interfered with, syneresis during storage is less likely, form retention is good, collapse in the oral cavity is unlikely, bad tastes such as bitter tastes and harsh tastes are suppressed, throat passage is good, and swallowing is easy, and which is suitable for routine ingestion. As a result, the inventors found that a gel preparation for oral administration having a pH level between 7 and 10, wherein syneresis is unlikely, storage stability is good, and bad tastes such as bitter tastes and harsh tastes are suppressed, can be obtained by blending a combination of ι carrageenan and locust bean gum as a gelling agent, in combination with one or more substances selected from agar, polysaccharides having a sulfuric acid group and/or a carboxyl group, cellulose derivatives, mannans, polysaccharides having an amino group, and starch derivatives, blending as required, one or more substances selected from cellulose derivatives, polyvinyl-series compounds, polysaccharides, starch derivatives, macrogol, alginic acid and derivatives thereof, as a polymer compound, and blending a salt of an organic and/or an inorganic acid and a cation and/or a hydroxide thereof as a buffering agent. And they developed the present invention.

Accordingly, the present invention relates to a gel preparation for oral administration comprising an HMG-CoA reductase inhibitor, a gelling agent, a polymer compound, a salt of an acid and a cation and/or a hydroxide thereof, a base, a sweetening agent, an antiseptic, and water, and having a pH level between 7 and 10.

### BEST MODE FOR CARRYING OUT THE INVENTION

The HMG-CoA reductase inhibitor contained as an active ingredient of the gel preparation for oral administration of the present invention is administered as a therapeutic agent for hyperlipemia, and may be derived from any of microbially-derived natural substances, semi-synthetic derivatives and totally synthetic compounds; examples include the statin-series compounds described in patent documents 1 to 8, preferably pravastatin, lovastatin, simvastatin, fluvastatin, livastatin, atorvastatin, rosvastatin or pitavastatin, more preferably pravastatin, lovastatin, simvastatin, fluvastatin, atorvastatin, rosvastatin or pitavastatin, still more preferably pravastatin, atorvastatin, fluvastatin or pitavastatin, and/or a pharmacologically acceptable salt thereof (suitably sodium salt or calcium salt and the like). Each HMG-CoA reductase inhibitor involves geometrical isomers, or, if containing asymmetric carbons, stereoisomers; these isomers can exist as hydrated form; all of these isomers and mixtures thereof are encompassed in the present invention.

[Patent document 1] JP-A-SHO-57-2240 (U.S. Pat. No. 4,346,227)
[Patent document 2] JP-A-SHO-57-163374 (U.S. Pat. No. 4,231,938)
[Patent document 3] JP-A-SHO-56-122375 (U.S. Pat. No. 444,784)
[Patent document 4] JP-T-SHO-60-500015 (U.S. Pat. No. 4,739,073)
[Patent document 5] JP-A-HEI-1-216974 (U.S. Pat. No. 5,006,530)
[Patent document 6] JP-A-HEI-1-279866 (U.S. Pat. No. 5,854,259 and U.S. Pat. No. 5,856,336)
[Patent document 7] JP-A-HEI-3-58967 (U.S. Pat. No. 5,273,995)
[Patent document 8] JP-A-HEI-5-178841 (U.S. Pat. No. 5,260,440)

In Japan, the usual dosage of an HMG-CoA reductase inhibitor in the gel preparation for oral administration of the present invention is about 1 to about 30 mg per administration, depending on the choice of HMG-CoA reductase inhibitor; for example, 5 to 10 mg for pravastatin sodium, 5 to 20 mg for simvastatin, 10 mg for atorvastatin, 10 to 30 mg for fluvastatin, and 1 to 2 mg for pitavastatin calcium. The weight of an easily ingestible (for example, ingestible at a mouthful) jelly of the present invention is usually about 0.4 to about 10 g. Hence, the content ratio of HMG-CoA reductase inhibitor is preferably 0.001 to 50% by mass, more preferably 0.01 to 40% by mass, still more preferably 0.05 to 20% by mass, relative to the total amount of the entire composition.

In the gel preparation for oral administration of the present invention, agar, pectin, sodium alginate, carrageenan, locust bean gum, xanthan gum, and guar gum can serve as gelling agents showing good gelling performance at pH levels between 7 and 10 and in the presence of an HMG-CoA reductase inhibitor during manufacturing of the preparation. However, if a gelling agent showing strong gelling power, such as agar or κ carrageenan, is used alone, syneresis during storage of the preparation is likely to occur, although the moldability is good. On the other hand, if a gelling agent showing weak or almost no gelling power, such as ι carrageenan, locust bean gum, or xanthan gum, is used alone, the moldability is poor, although the syneresis is very unlikely to occur. In both cases, the preparations are not suitable for taking orally. These shortcomings have been effectively improved by using in combination two or more gelling agents selected from agar, non-ionic polysaccharides, polysaccharides having a sulfuric acid group and/or a carboxyl group, aminopolysaccharides, cellulose derivatives and proteins. More specifically, such combinations of gelling agents include combinations of two or more substances selected from gelatin, milk casein, mannans, carrageenan, starch, gelatinized starch, tragacanth gum, tamarind seed polysaccharide, gellan gum, karaya gum, cassia gum, tara gum, chitosan, psyllium seed gum, ghatti gum and the like, exemplified by a combination of agar, ι carrageenan, xanthan gum and locust bean gum, a combination of κ carrageenan, locust bean gum, guar gum and pectin, a combination of ι carrageenan, κ carrageenan, locust bean gum and guar gum, and a combination of alginic acid sodium, pectin, xanthan gum and agar. The amount of these gelling agents added is preferably 0.05 to 15% by mass, more preferably 0.2 to 10% by mass, still more preferably 0.5 to 5% by mass, relative to the total amount of the entire gel preparation.

Adding a polymer compound to increase the gel form retenting capability of the gelling agent contained in the gel preparation of the present invention is effective to decrease syneresis and leads to mitigate the bitter and harsh tastes of the HMG-CoA reductase inhibitor to improve the sensory property by enhancing the gel form retention in the oral cavity at the time of ingestion. The polymer compound added to provide this function is selected from cellulose derivatives, polyvinyl-series compounds, polysaccharides, starch derivatives, macrogol, alginic acid and derivatives thereof, hydroxypropylcellulose, hydroxyethylmethylcellulose, methylcellulose and the like, and these can be used singly or in combination. The amount of these polymers added is preferably 0.05 to 15% by mass, more preferably 0.2 to 10% by mass, still more preferably 0.5 to 5% by mass, relative to the total amount of the entire gel preparation.

Buffering agents that are effective in keeping the gel preparation for oral administration of the present invention at a pH between 7 and 10, and that produce a gel showing almost no syneresis, include salts of organic acids such as lactic acid, citric acid, tartaric acid, malic acid, and adipic acid and/or inorganic acids such as phosphoric acid, and metal ions such as potassium ion, sodium ion, calcium ion and magnesium ion and/or hydroxides of these metal ions. The amount of buffering agent added is not subject to limitation, and is preferably not less than 0.001% by mass and not more than 15% by mass, relative to the total amount of the entire gel preparation.

The gel preparation for oral administration of the present invention may contain preservative agents. Examples of the preservative agents include isobutyl para-oxybenzoate, isopropyl para-oxybenzoate, ethyl para-oxybenzoate, propyl para-oxybenzoate, methyl para-oxybenzoate, benzoic acid, sodium edetate, calcium disodium edetate, salicylic acid, sorbic acid, dehydroacetic acid and salts thereof, chlorobutanol, benzyl alcohol, phenylethyl alcohol, phenoxyethanol, 1-menthol, dl-camphor, cetylpyridinium chloride, benzethonium chloride, benzalkonium chloride and the like; the amount of antiseptic added is not subject to limitation.

The base contained in the gel preparation for oral administration of the present invention is exemplified by polyhydric alcohol glycerols, propylene glycol, D-sorbitol, xylitol, mannitol, erythritol, maltitol, and saccharides such as trehalose, raffinose, sucrose, fructose, glucose, and lactose; and the amount of substrate added is not subject to limitation.

The sweetening agent contained in the gel preparation for oral administration of the present invention is exemplified by saccharin sodium, aspartame, stevia, glycyrrhizinic acid, derivatives thereof, and mixtures thereof; these sweetening agents are particularly used to mitigate the bitter tastes and harsh tastes of HMG-CoA reductase inhibitor adjusted to a pH levels between 7 and 10; the ratio by weight of the HMG-CoA reductase inhibitor to the sweetening agent is 1:0.001 to 1:40. Furthermore, appropriate amounts of fructose, purified white soft sugar, palatinose, trehalose, oligosaccharides, isomerized sugars, muscovado, hydrangea macrophyla powder, glycyrrhiza extract, sucralose, thaumatin, glucose, starch syrup, reducing maltose starch syrup and the like may be used to improve the taste of the gel preparation.

The gel preparation for oral administration of the present invention can be prepared in the same manner as the method for preparing a commonly known gel composition except that the above-described ingredients may be blended. More specifically, an HMG-CoA reductase inhibitor and other ingredients are added to an appropriate amount of water or warm water, this mixture is uniformly stirred and homogenized optionally with heating using a mechanical stirrer equipped with an appropriate mechanism for heating and cooling; the resulting homogenate is filled and cooled to solidify in an optionally chosen container, whereby the gel preparation for oral administration of the present invention can be prepared.

Although the gelling time varies depending on the amount of drug liquid filled, the drug liquid usually gels to yield a gel composition of the present invention when allowed to stand at normal temperature for 1 to 2 hours, or at 10° C. or lower for 1 hour.

Although the manufacturing process comprises the following steps, the individual raw materials may not always be added in the order indicated, and the order can be changed as appropriate. The HMG-CoA reductase inhibitor used in step (2) is exemplified by pravastatin sodium, fluvastatin sodium, atorvastatin calcium hydrate, pitavastatin calcium, simvastatin and the like.

(1) Weigh out water and a buffering agent, place in the preparation chamber, and the mixture is stirred and dissolved at room temperature or under appropriate heating.
(2) Add an HMG-CoA reductase inhibitor.
(3) Add a gelling agent and a polymer compound, and the mixture is stirred and dissolved with heating at 75 to 95° C.
(4) Add a preservative agent, a flavoring agent, a sweetening agent and the like, and thereafter adjust the pH levels to 7 to 10, and perform thermal sterilization.
(5) While maintaining an appropriate temperature, dispense the drug liquid from the preparation chamber to an optionally chosen container.
(6) Cool the drug liquid to solidify using an appropriate cooling apparatus or method, and package to obtain a finished product.

### EFFECT OF THE INVENTION

As is evident from the results of the evaluations of the formulations shown in Examples 1 to 8 and Comparative Examples 1 to 8, gel preparations for oral administration comprising a gelling agent, a polymer compound, a buffering agent, a sweetening agent, a preservative agent, a base water and a compound belonging to the HMG-CoA reductase inhibitor having a pH levels between 7 and 10 were found to exhibit good gel form retention, to be unlikely to undergo syneresis, to have excellent storage stability, and to have suppressed bad tastes such as bitter tastes and harsh tastes.

The present invention is described in more detail by means of the following examples, which, however, are not to be construed as limiting the scope of the present invention.

### EXAMPLES

Gel preparations for oral administration were prepared as described in Example 1, using the formulations shown in Examples 1 to 8 and Comparative Examples 1 to 8 (e.g., 10.53 to 31.59 mg of fluvastatin sodium, 1 to 2 mg of pitavastatin, and 5 to 10 mg of pravastatin sodium were given each time).

All example formulations for gel preparations for oral administration comprising an HMG-CoA reductase inhibitor as the pharmacologically active ingredient, are shown in Tables 1 to 4. Each formulation was filled up to a container with a capacity of 1 to 10 mL, and the container was closed and cooled to yield a gel preparation for oral administration. The gel preparations for oral administration thus obtained had a pH levels between 7 and 10.

TABLE 1

| | | Example formulations (% by mass) | | | |
|---|---|---|---|---|---|
| | | | | Comp. | Comp. |
| | | Example 1 | Example 2 | Example 1 | Example 2 |
| Fluvastatin sodium | | 0.7 | 0.7 | 0.7 | 0.7 |
| base/addi- | Pectin | 0.1 | 0 | 0 | 0 |
| tives | Locust bean | 0.3 | 0.2 | 0 | 0.3 |
| | gum | | | | |
| | Xanthan gum | 0.1 | 0 | 0 | 0 |
| | κ carrageenan | 0.2 | 0.1 | 0 | 0 |
| | ι carrageenan | 0.4 | 0.6 | 0 | 0.4 |
| | Agar powder | 0 | 0.3 | 0.8 | 0 |
| | Microcrys- | 0 | 0 | 0 | 0 |
| | talline | | | | |
| | cellulose | | | | |
| | D-sorbitol | 20.0 | 20.0 | 20.0 | 20.0 |
| | Glycerol | 10.0 | 10.0 | 10.0 | 10.0 |
| | Saccharin | 0.1 | 0.1 | 0.1 | 0.1 |
| | sodium | | | | |
| | Sodium | 1.0 | 0 | 0 | 1.0 |
| | citrate | | | | |
| | Citric acid | 0.001 | 0.5 | 0.5 | 0.001 |
| | Sodium | 0 | 0.3 | 0.3 | 0 |
| | hydroxide | | | | |
| | Ethyl | 0.04 | 0.04 | 0.04 | 0.04 |
| | paraben | | | | |
| | Propyl | 0.02 | 0.02 | 0.02 | 0.02 |
| | paraben | | | | |
| | Strawberry | 0.05 | 0.05 | 0.05 | 0.05 |
| | essence | | | | |
| | Purified | Remainder | Remainder | Remainder | Remainder |
| | water | | | | |
| Total | | 100 | 100 | 100 | 100 |
| pH | | 8.3 | 9.2 | 8.9 | 8.4 |
| Evaluation | Gel form | Good | Good | Good | Poor |
| | retention | | | | |
| | Syneresis | Almost no | Almost no | Intensified | Almost no |
| | during | syneresis | syneresis | over time | syneresis |
| | storage at | observed | observed | | observed |
| | room | | | | |
| | temperature | | | | |
| | Ease of | Easy to | Easy to | Liquid | Large |
| | removal | remove, and | remove, | spills and | amount of |
| | from | small | and small | difficult | liquid |
| | container | amount of | amount of | to remove | remaining |
| | | liquid | liquid | | and |
| | | remaining | remaining | | difficult |
| | | | | | to remove |
| | Tastes upon | No bad | No bad | Uncomfort- | Uncomfort- |
| | ingestion | tastes such | tastes | able | able |
| | | as bitter | such as | tastes | tastes |
| | | tastes and | bitter | such as | such as |
| | | harsh | tastes and | bitter | bitter |
| | | tastes | harsh | tastes and | tastes and |
| | | perceived | tastes | harsh | harsh |
| | | | perceived | tastes | tastes |
| | | | | perceived | perceived |
| | | | | slightly | |

TABLE 2

| | | Example formulations (% by mass) | | | |
|---|---|---|---|---|---|
| | | | | Comp. | Comp. |
| | | Example 3 | Example 4 | Example 3 | Example 4 |
| Pitavastatin calcium | | 0.05 | 0.05 | 0.05 | 0.05 |
| base/addi- | Pectin | 0 | 0.1 | 0 | 0 |
| tives | Locust bean | 0.3 | 0.25 | 0 | 0 |
| | gum | | | | |
| | Xanthan gum | 0.4 | 0 | 0.5 | 0 |
| | κ carrageenan | 0 | 0.2 | 0 | 0.6 |
| | ι carrageenan | 0.6 | 0.55 | 0 | 0 |
| | Agar powder | 0.1 | 0 | 0 | 0 |
| | Microcrys- | 0.2 | 0.3 | 0.2 | 0.4 |
| | talline | | | | |
| | cellulose | | | | |
| | D-sorbitol | 10.0 | 10.0 | 10.0 | 10.0 |
| | Erythritol | 15.0 | 15.0 | 15.0 | 15.0 |
| | Glycerol | 5.0 | 5.0 | 5.0 | 5.0 |
| | Saccharin | 0.1 | 0.1 | 0.1 | 0.1 |
| | sodium | | | | |
| | Disodium | 1.0 | | 0.8 | 0 |
| | hydrogen | | | | |
| | phosphate | | | | |
| | Phosphoric | 0.01 | 0.6 | 0.005 | 0.5 |
| | acid | | | | |
| | Sodium | 0 | 0.5 | 0 | 0.4 |
| | hydroxide | | | | |
| | Ethyl paraben | 0 | 0.02 | 0.02 | 0.04 |
| | Propyl | 0.04 | 0.02 | 0.02 | 0.04 |
| | paraben | | | | |
| | 1-menthol | 0.04 | 0.04 | 0.04 | 0.04 |
| | Purified | Remainder | Remainder | Remainder | Remainder |
| | water | | | | |
| Total | | 100 | 100 | 100 | 100 |
| pH | | 8.0 | 9.0 | 7.8 | 8.7 |
| Evaluation | Gel form | Good | Good | Poor | Good |
| | retention | | | | |
| | Syneresis | Almost no | Almost no | Almost no | Intensified |
| | during storage | syneresis | syneresis | syneresis | over time |
| | at room | observed | observed | observed | |
| | temperature | | | | |
| | Ease of | Easy to | Easy to | Large | Liquid |
| | removal from | remove, | remove, | amount of | spills and |
| | container | and small | and small | liquid | difficult |
| | | amount of | amount of | remaining | to remove |
| | | liquid | liquid | and | |
| | | remaining | remaining | difficult | |
| | Tastes upon | No bad | No bad | Bad tastes | Bad tastes |
| | ingestion | tastes | tastes | such as | such as |
| | | such as | such as | bitter | bitter |
| | | bitter | bitter | tastes and | tastes and |
| | | tastes and | tastes and | harsh | harsh |
| | | harsh | harsh | tastes | tastes |
| | | tastes | tastes | perceived | perceived |
| | | perceived | perceived | slightly | slightly |

TABLE 3

| | | Example formulations (% by mass) | | | |
|---|---|---|---|---|---|
| | | | | Comp. | Comp. |
| | | Example 5 | Example 6 | Example 5 | Example 6 |
| Pravastatin sodium | | 0.25 | 0.25 | 0.25 | 0.25 |
| base/addi- | Pectin | 0.2 | 0 | 0 | 0 |
| tives | Locust bean | 0.2 | 0.2 | 0.2 | 0 |
| | gum | | | | |
| | Xanthan gum | 0.4 | 0.1 | 0 | 0 |
| | κ carrageenan | 0 | 0.1 | 0 | 0 |
| | ι carrageenan | 0.5 | 0.6 | 0.5 | 0.5 |
| | Agar powder | 0 | 0.1 | 0 | 0.5 |
| | Microcrys- | 0 | 0 | 0 | 0 |
| | talline | | | | |
| | cellulose | | | | |
| | Powdered | 15.0 | 10.0 | 10.0 | 10.0 |
| | reducing | | | | |
| | maltose | | | | |
| | starch syrup | | | | |
| | Erythritol | 0 | 10.0 | 10.0 | 0 |
| | Glycerol | 15.0 | 10.0 | 10.0 | 15.0 |
| | Saccharin | 0.2 | 0.15 | 0.15 | 0.2 |
| | sodium | | | | |
| | Sodium | 0.8 | 0.8 | 0 | 0.8 |
| | citrate | | | | |
| | Malic acid | 0.01 | 0.01 | 0.5 | 0 |
| | Sodium | 0 | 0 | 0.4 | 0 |
| | hydroxide | | | | |
| | Ethyl paraben | 0.02 | 0.02 | 0.02 | 0.02 |
| | Propyl | 0.02 | 0.02 | 0.02 | 0.02 |
| | paraben | | | | |
| | 1-menthol | 0.08 | 0.05 | 0.05 | 0.08 |
| | Purified | Remainder | Remainder | Remainder | Remainder |
| | water | | | | |
| Total | | 100 | 100 | 100 | 100 |
| pH | | 8.3 | 8.3 | 9.2 | 8.4 |
| Evaluation | Gel form | Good | Good | Poor | Good |
| | retention | | | | |
| | Syneresis | Almost no | Almost no | Almost no | Intensified |
| | during storage | syneresis | syneresis | syneresis | over time |
| | at room | observed | observed | observed | |
| | temperature | | | | |
| | Ease of | Easy to | Easy to | Large | Liquid |
| | removal from | remove, | remove, | amount of | spills and |
| | container | and small | and small | liquid | difficult |
| | | amount of | amount of | remaining | to remove |
| | | liquid | liquid | and | |
| | | remaining | remaining | difficult | |
| | | | | to remove | |
| | Tastes upon | No bad | No bad | Bad tastes | Bad tastes |
| | ingestion | tastes | tastes | such as | such as |
| | | such as | such as | bitter | bitter |
| | | bitter | bitter | tastes and | tastes and |
| | | tastes and | tastes and | harsh | harsh |
| | | harsh | harsh | tastes | tastes |
| | | tastes | tastes | perceived | perceived |
| | | perceived | perceived | | |

TABLE 4

| | | Example formulations (% by mass) | | | |
|---|---|---|---|---|---|
| | | | | Comp. | Comp. |
| | | Example 7 | Example 8 | Example 7 | Example 8 |
| Atorvastatin calcium | | 0.36 | 0.36 | 0.36 | 0.36 |
| hydrate | | | | | |
| base/addi- | Pectin | 0 | 0.2 | 0.4 | 0 |
| tives | Locust bean | 0.2 | 0.2 | 0 | 0 |
| | gum | | | | |
| | Xanthan gum | 0.2 | 0.3 | 0 | 0 |
| | κ carrageenan | 0 | 0.15 | 0 | 0.4 |
| | ι carrageenan | 0.6 | 0.6 | 0.4 | 0 |
| | Agar powder | 0.2 | 0 | 0 | 0.4 |
| | Microcrys- | 0.5 | 0.8 | 0.8 | 0.5 |
| | talline | | | | |
| | cellulose | | | | |
| | Trehalose | 15.0 | 10.0 | 15.0 | 10.0 |
| | Xylitol | 15.0 | 10.0 | 15.0 | 10.0 |
| | Glycerol | 0 | 10.0 | 0 | 10.0 |
| | Saccharin | 0.1 | 0.1 | 0.1 | 0.1 |
| | sodium | | | | |
| | Sodium | 1.2 | 0 | 0 | 0.8 |
| | citrate | | | | |
| | Citric acid | 0.01 | 0.6 | 0.5 | 0 |
| | Sodium | 0 | 0.5 | 0.4 | 0 |
| | hydroxide | | | | |
| | Ethyl | 0.03 | 0.04 | 0.04 | 0.03 |
| | paraben | | | | |
| | Propyl | 0.03 | 0.02 | 0.02 | 0.03 |
| | paraben | | | | |
| | Lemon | 0.04 | 0.04 | 0.04 | 0.04 |
| | essence | | | | |
| | Purified | Remainder | Remainder | Remainder | Remainder |
| | water | | | | |
| Total | | 100 | 100 | 100 | 100 |
| pH | | 7.8 | 9.2 | 8.6 | 8.3 |
| Evaluation | Gel form | Good | Good | Poor | Good |
| | retention | | | | |
| | Syneresis | Almost no | Almost no | Almost no | Intensified |
| | during | syneresis | syneresis | syneresis | over time |
| | storage at | observed | observed | observed | |
| | room | | | | |
| | temperature | | | | |
| | Ease of | Easy to | Easy to | Large | Liquid |
| | removal | remove, and | remove, | amount of | spills and |
| | from | small | and small | liquid | difficult |
| | container | amount of | amount of | remaining | to remove |
| | | liquid | liquid | and | |
| | | remaining | remaining | difficult | |
| | | | | to remove | |
| | Tastes upon | No bad | No bad | Bad tastes | Bad tastes |
| | ingestion | tastes such | tastes | such as | such as |
| | | as bitter | such as | bitter | bitter |
| | | tastes and | bitter | tastes and | tastes and |
| | | harsh | tastes and | harsh | harsh |
| | | tastes | harsh | tastes | tastes |
| | | perceived | tastes | perceived | perceived |
| | | | perceived | slightly | slightly |

### INDUSTRIAL APPLICABILITY

A gel preparation for oral administration that is easily swallowable and eliminates the bad tastes such as bitter tastes and harsh tastes of the HMG-CoA reductase inhibitor contained therein can be provided for patients with hyperlipemia or hypercholesterolemia in need of oral administration of an HMG-CoA reductase inhibitor by enclosing or packaging the gel preparation in containers that are easily handleable at the time of ingestion, whereby the drug compliance of patients with these diseases, including those with difficulty in taking conventional preparations, are significantly improved.

1. A gel preparation for oral administration comprising an HMG-CoA reductase inhibitor, a gelling agent, a polymer compound, a buffering agent, a sweetening agent, a base and water, and having a pH level between 7 and 10.

2. The gel preparation for oral administration according to claim 1, wherein the HMG-CoA reductase inhibitor is pravastatin, lovastatin, simvastatin, fluvastatin, livastatin, atorvastatin, rosvastatin, pitavastatin or a pharmacologically acceptable salt thereof.

3. The gel preparation for oral administration according to claim 1, wherein the gelling agent is a combination of carrageenan, locust bean gum, and one or more substances selected from agar, non-ionic polysaccharides, polysaccharides having a sulfuric acid group and/or a carboxyl group, aminopolysaccharides, cellulose derivatives and proteins.

4. The gel preparation for oral administration according to claim 1, wherein the polymer compound is used in combination with one or more water-soluble polymers selected from cellulose derivatives, polyvinyl-series compounds, polysaccharides, starch derivatives, macrogol, alginic acid and derivatives thereof.

5. The gel preparation for oral administration according to claim 1, wherein the buffering agent consists of a combination of a salt of an organic acid such as lactic acid, citric acid, tartaric acid, malic acid, or adipic acid and/or an inorganic acid such as phosphoric acid and a metal ion such as potassium ion, sodium ion, calcium ion or magnesium ion and/or a hydroxide of one of these metal ions.

6. The gel preparation for oral administration according to claim 1, wherein the sweetening agent is one selected from saccharin sodium, stevia, aspartame, glycyrrhizinic acid and derivatives thereof or a combination of two or more thereof, and wherein the ratio by weight of the HMG-CoA reductase inhibitor to the sweetening agent is 1:0.001 to 1:40.
